(19) United States
(12) Patent Application Publication
(10) Pub. No.: US 2025/0082370 A1
(43) Pub. Date: Mar. 13, 2025

(54) REVISION SYSTEM FOR SPINAL IMPLANTS

(71) Applicant: Nexus Spine, LLC, Salt Lake City, UT (US)

(72) Inventors: David Hawkes, Salt Lake City, UT (US); Peter Halverson, Salt Lake City, UT (US); Eric Dodgen, Salt Lake City, UT (US); Quentin Aten, Salt Lake City, UT (US)

(21) Appl. No.: 18/727,569

(22) PCT Filed: Feb. 16, 2017
(86) PCT No.: PCT/US17/18244

§ 371 Date: Jul. 9, 2024

Related U.S. Application Data
(60) Provisional application No. 62/296,072, filed on Feb. 16, 2016.

(30) Foreign Application Priority Data
Feb. 16, 2016 (WO) PCT/US17/18244

(51) Int. Cl. A61B 17/70; A61B 17/00; A61B 17/56

(52) U.S. Cl. CPC A61B 17/705; A61B 17/7052; A61B 2017/00477; A61B 2017/00991; A61B 2017/564

(57) ABSTRACT

Systems and methods for spinal fusion revision allow extending prior spinal fusion constructs in revision surgery, in situ, while minimizing surgical exposure of the prior constructs. A revision system for extending spinal fusion implants includes a tulip assembly adapted to be fixedly secured to a pedicle screw as part of a first spinal fusion procedure and a construct extension protrusion extending from the tulip assembly. A revision system for extending spinal fusion implants in situ includes a tulip assembly adapted to be fixedly secured to a pedicle screw as part of a second spinal fusion procedure adjacent a site of a prior spinal fusion procedure. The tulip assembly includes a receptacle adapted to be fixedly secured to a construct extension protrusion of a spinal fusion construct from the prior spinal fusion procedure via a press or interference fit.

### TECHNICAL FIELD

The present invention relates to spinal implants, and more particularly to a system for facilitating revision of spinal fusion surgery.

### BACKGROUND ART

Spinal lumbar fusion surgery triggers a cascade of adjacent spinal segment degeneration. The levels above and/or below a fused level can become painfully symptomatic within a few years. Subsequent fusion of an adjacent level can be difficult because the already-fused level still needs hardware to support the relatively weak bone of the fusion mass. However, the already-implanted hardware precludes the implantation of new hardware at the adjacent level.

Removing the already-implanted hardware and then replacing it with a longer construct is onerous and risky. So, some have created extending attachments that allow the already implanted hardware to stay in place; but, the predicate extending attachments are very bulky and are difficult to use. For example, FIG. 1 illustrates an exemplary prior extending attachment 10 adapted to be affixed to a rod 12 of an existing implant assembly via a clamping assembly 14. As may be appreciated from FIG. 1, the clamping assembly 14 must extend posteriorly approximately twice the maximal posterior extension of the existing implant assembly so as to pass over an existing pedicle screw assembly 16 from the existing implant assembly. Accordingly, implantation of the extending attachment 10 results in a very bulky final assembly at the level of the existing implant adjacent where the existing implant was extended. Additionally, such extending attachments require re-exposing much of the original hardware, which is painful and time-consuming.

Because in many instances such implants are used bilaterally, the problems discussed above are commonly duplicated on each side of the spine during a fixation procedure. As a result, existing spinal fusion systems used for extension of an existing fusion fail to adequately address the unique needs of revision surgeries.

### SUMMARY OF THE INVENTION

Implementation of the invention provides systems and methods for extending prior spinal fusion constructs in revision surgery while minimizing surgical exposure of the prior constructs. According to implementations of the invention, a revision system for extending spinal fusion implants in situ includes a tulip assembly adapted to be fixedly secured to a pedicle screw as part of a first spinal fusion procedure and a construct extension protrusion extending from the tulip assembly in a direction roughly parallel to a next level of a spine adjacent to a level of the spine to be fused as part of the first spinal fusion procedure. The construct extension protrusion may be integrally formed with the tulip assembly.

The construct extension protrusion may extend at an angle of within twenty degrees of parallel to the next level of the spine. Alternatively, the construct extension protrusion may extend at an angle of within ten degrees of parallel to the next level of the spine. The construct extension protrusion may include or have a cylindrical shape. The construct extension protrusion may have or include a spherical portion or partially-spherical portion.

The revision system may also include an additional tulip assembly adapted to be fixedly secured to an additional pedicle screw as part of a second spinal fusion procedure. The additional tulip assembly may include a receptacle adapted to be fixedly secured to the construct extension protrusion via a press or interference fit. The receptacle may include a slot that permits the receptacle to flex open or closed. The additional tulip assembly may also include a cinch band adapted to be moved over a portion of the receptacle including the slot so as to force the receptacle to a closed position forming the press or interference fit over the construct extension protrusion. The revision system may further include a slotted ball adapted to be placed over the construct extension protrusion and to fit within the receptacle as the press or interference fit is achieved.

Before the interference or press fit is achieved, the construct extension protrusion may be received within the receptacle while a range of motion is maintained between the construct extension protrusion and the additional tulip assembly. The range of motion may include either or both of poly-axial rotation and flexion and telescopic motion of the construct extension protrusion within the receptacle. Once a final position is achieved, the motion between the construct extension protrusion and the receptacle is fixed by achieving the interference or press fit, e.g., by moving the cinch band over the portion of the receptacle including the slot.

According to further implementations of the invention, a revision system for extending spinal fusion implants in situ includes a tulip assembly adapted to be fixedly secured to a pedicle screw as part of a second spinal fusion procedure adjacent a site of a prior spinal fusion procedure. The tulip assembly includes a receptacle adapted to be fixedly secured to a construct extension protrusion of a spinal fusion construct from the prior spinal fusion procedure via a press or interference fit.

The receptacle may include a slot that permits the receptacle to flex open or closed. The tulip assembly may include a cinch band adapted to be moved over a portion of the receptacle having the slot so as to force the receptacle to a closed position forming the press or interference fit over the construct extension protrusion.

The revision system may also include a slotted ball adapted to be placed over the construct extension protrusion and to fit within the receptacle as the press or interference fit is achieved. Before the interference or press fit is achieved, the construct extension protrusion may be received within the receptacle while a range of motion is maintained between the construct extension protrusion and the tulip assembly. The maintained motion may include poly-axial rotation and flexion and/or telescopic motion of the construct extension protrusion within the receptacle. Once a final position is achieved, the motion between the construct extension protrusion and the receptacle is fixed by achieving the interference or press fit, e.g., by moving the cinch band over the portion of the receptacle including the slot.

According to further implementations of the invention, a method for performing spinal fusion to reduce surgical exposure required for any necessary future revision surgery includes assembling in situ a spinal fusion construct adapted to fuse one or more levels of the spine, wherein, upon assembly, a terminal end of the construct includes a construct extension protrusion extending from the construct in a direction roughly parallel to a next level of a spine adjacent to a level of the spine fused as part of a spinal fusion procedure in which the spinal fusion construct is used.

The construct extension protrusion may extend at an angle of within twenty degrees of parallel to the next level of the spine. Alternatively, the construct extension protrusion may extend at an angle of within ten degrees of parallel to the next level of the spine. The construct extension protrusion may include a cylindrical shape. The construct extension protrusion may include a spherical portion or a partially spherical portion.

According to further implementations of the invention, a method for performing spinal fusion while reducing surgical exposure at a site of a previous spinal fusion construct includes exposing a terminal end of the previous spinal fusion construct, the previous spinal fusion construct including a construct extension protrusion extending from the construct in a direction roughly parallel to a next level of a spine adjacent to a level of the spine fused as part of a prior spinal fusion procedure, securing a pedicle screw to a next pedicle of the spine, and securing an extension construct between the pedicle screw and the construct extension protrusion, wherein the extension construct is secured to the construct extension protrusion via a press fit or interference fit.

### BRIEF DESCRIPTION OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows a prior-art revision system;

FIG. 2 shows a side view of an embodiment of a revision system;

FIG. 3 shows a perspective view of an embodiment of a revision system;

FIG. 4 shows a top view of an embodiment of a revision system;

FIG. 5 shows an exploded side view of an embodiment of a revision system;

FIG. 6 shows an exploded perspective view of an embodiment of a revision system;

FIG. 7 shows a perspective view of an embodiment of a revision system;

FIG. 8 shows a perspective exploded view of a spinal construct adapted for later revision; and

FIG. 9 shows a perspective exploded view of an alternate spinal construct adapted for later revision.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

Embodiments of the invention provide systems and methods for extending prior spinal fusion constructs in revision surgery while minimizing surgical exposure of the prior constructs. According to embodiments of the invention, a revision system for extending spinal fusion implants in situ includes a tulip assembly adapted to be fixedly secured to a pedicle screw as part of a first spinal fusion procedure and a construct extension protrusion extending from the tulip assembly in a direction roughly parallel to a next level of a spine adjacent to a level of the spine to be fused as part of the first spinal fusion procedure. The construct extension protrusion may be integrally formed with the tulip assembly.

According to further embodiments of the invention, a revision system for extending spinal fusion implants in situ includes a tulip assembly adapted to be fixedly secured to a pedicle screw as part of a second spinal fusion procedure adjacent a site of a prior spinal fusion procedure. The tulip assembly includes a receptacle adapted to be fixedly secured to a construct extension protrusion of a spinal fusion construct from the prior spinal fusion procedure via a press or interference fit.

According to further embodiments of the invention, a method for performing spinal fusion to reduce surgical exposure required for any necessary future revision surgery includes assembling in situ a spinal fusion construct adapted to fuse one or more levels of the spine, wherein, upon assembly, a terminal end of the construct includes a construct extension protrusion extending from the construct in a direction roughly parallel to a next level of a spine adjacent to a level of the spine fused as part of a spinal fusion procedure in which the spinal fusion construct is used.

According to further embodiments of the invention, a method for performing spinal fusion while reducing surgical exposure at a site of a previous spinal fusion construct includes exposing a terminal end of the previous spinal fusion construct, the previous spinal fusion construct comprising a construct extension protrusion extending from the construct in a direction roughly parallel to a next level of a spine adjacent to a level of the spine fused as part of a prior spinal fusion procedure, securing a pedicle screw to a next pedicle of the spine, and securing an extension construct between the pedicle screw and the construct extension protrusion, wherein the extension construct is secured to the construct extension protrusion via a press fit or interference fit.

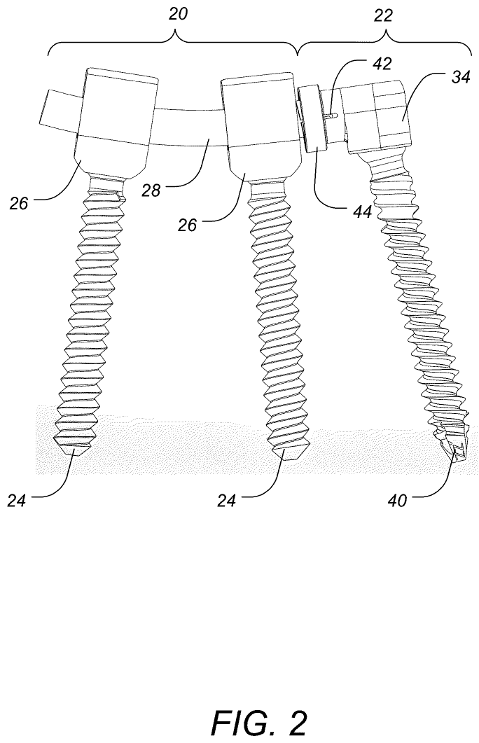

While FIGS. 2-7 show various views of illustrative embodiments of revision systems embodying the principles discussed herein, it should be recognized that the illustrated embodiments are intended to be merely illustrative of the principles discussed herein. Specifically, it should be noted that the various embodiments of surgical constructs illustrated in the Related Applications could be modified to include construct extension protrusions in like manner as is shown in FIGS. 2-7, and all of the advantages achieved and disclosed in the Related Applications could be achieved while also providing the revision advantages provided by the embodiments illustrated herein.

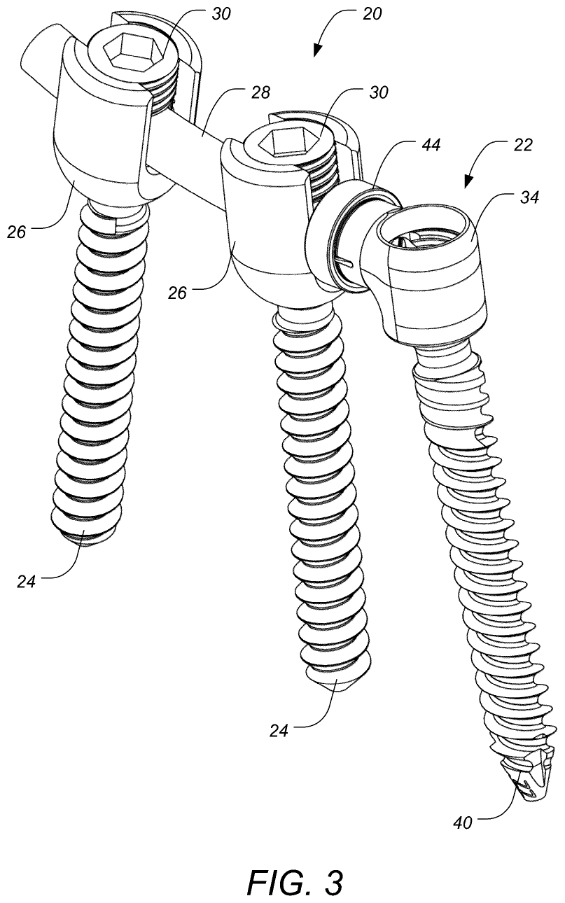

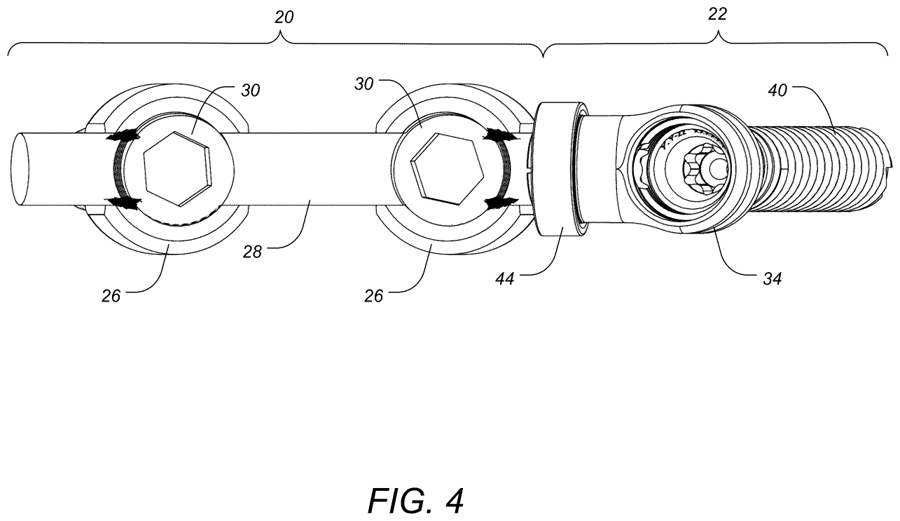

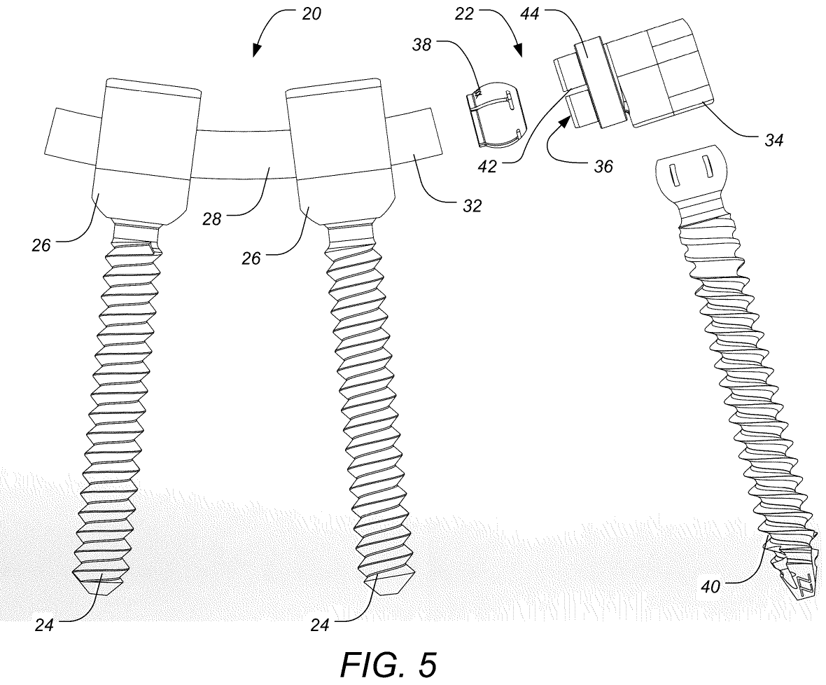

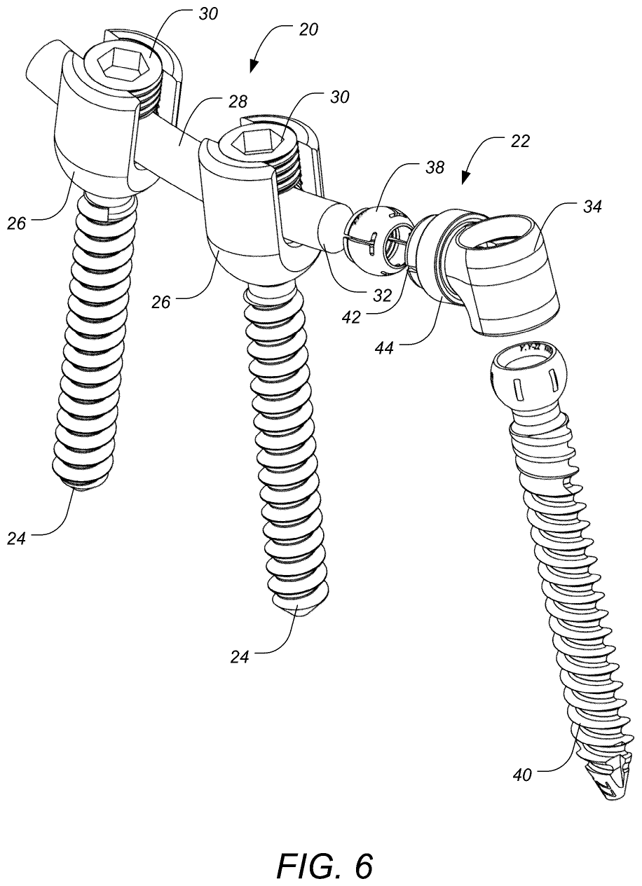

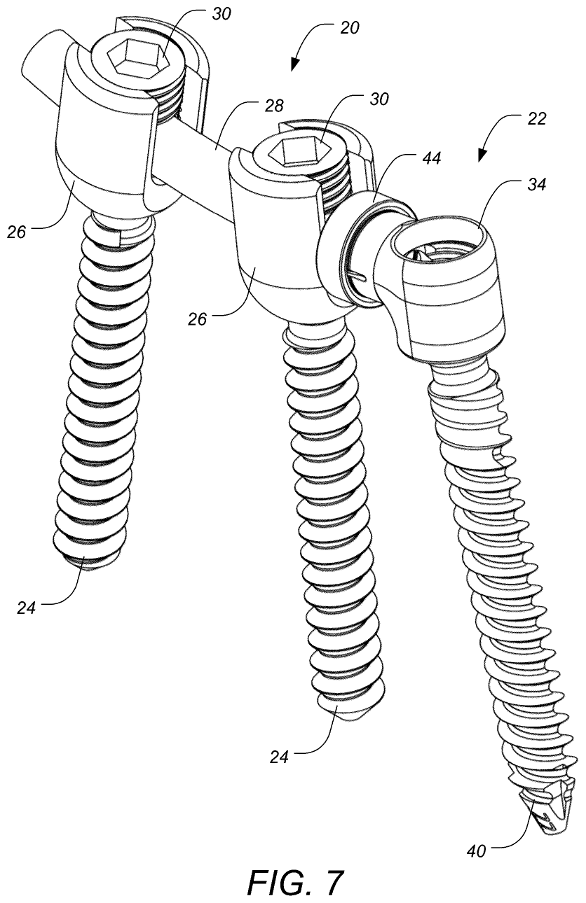

FIGS. 2-7 illustrate various views of a first spinal fusion construct 20 and of a revision spinal fusion construct extender 22. FIG. 2 shows a side view of the first spinal fusion construct 20 assembled with the revision spinal fusion construct extender 22. FIGS. 3 and 7 show perspective views of the first spinal fusion construct 20 assembled with the revision spinal fusion construct 22. FIG. 4 shows a top view of the first spinal fusion construct 20 assembled with the revision spinal fusion construct 22. FIG. 5 shows a side exploded view of the first spinal fusion construct 20 and the revision spinal fusion construct 22. FIG. 6 shows a perspective exploded view of the first spinal fusion construct 20 and the revision spinal fusion construct 22.

The first spinal fusion construct 20 is generally implanted into a patient as part of a spinal fusion procedure for fusing one or more levels of the spine (the embodiment of FIGS. 2-7 show a first spinal fusion construct 20 for a single level fusion). The first spinal fusion construct includes two pedicle screws 24 that would be implanted into the pedicles of adjacent vertebra (not shown in FIGS. 2-7). A first construct tulip assembly 26 is assembled to each pedicle screw 24, using methods known in the art. Typical devices for affixing tulip assemblies 26 to the pedicle screws 24 involve a system where the pedicle screw 24 is inserted into the tulip assembly 26 prior to implanting the screw 24, whereupon additional elements of the construct 20 are assembled to the tulip assemblies 26 to fix the position of the tulip assemblies 26 on the pedicle screws 24 and relative to each other. The assembly typically depends on the use of a rod 28 extending between tulip assemblies 26, as well as a set screw 30 for each tulip assembly 26. The set screw 30 tightens the rod 28 down into the tulip assembly 26, which simultaneously presses the rod 28 down on the top of the pedicle screw 24, locking the entire assembly.

The Related Applications discuss deficiencies inherent with this style of spinal fusion construct, and further disclose alternate constructs that more efficiently and compactly provide many of the same functions as the old style of spinal fusion construct. Nevertheless, the rod 28 can be used in conjunction with embodiments of the revision spinal fusion construct 22 to provide a more compact revision fusion to adjacent spinal levels. As discussed above, it is common for fusion of one or more levels of the spine to eventually result in adjacent spinal segment degeneration. The revision spinal fusion construct 22 permits a revision surgery to fuse adjacent spinal segments and add additional fusion hardware without exposing the entire first spinal fusion construct 20, and without requiring bulky additions to the original first spinal fusion construct 20.

Instead, the revision spinal fusion construct 22 uses a construct extension protrusion 32 of the first spinal fusion construct 20, which may be a protruding portion of the rod 28. The construct extension protrusion may extend in a direction roughly parallel to the next segment or level of the spine, such as within approximately twenty degrees of parallel or within approximately ten degrees of parallel. This is best seen in the exploded views of FIGS. 5 and 6. The revision spinal fusion construct 22 includes a revision tulip assembly 34 that includes a receptacle 36 (an example of a female member) adapted to receive the construct extension protrusion 32 therein, either directly or with a slotted ball 38 that is sized and adapted to compress within the receptacle 36 and over the construct extension protrusion 32 as a press or interference fit is created between the construct extension protrusion 32 and the receptacle 36. The revision tulip assembly 34 may include a screw receptacle adapted to receive a revision pedicle screw 40 via a press or interference fit as disclosed in the Related Applications and as shown in FIGS. 2-7.

The receptacle 36 may include a slot 42 that permits the receptacle 36 to expand as it receives the construct extension protrusion 32 (and, if present, the slotted ball 38), but to be compressed to fixedly secure the construct extension protrusion 32 (and the slotted ball 38). This compression may be effected by a cinch band 44 adapted to slide back and forth along the exterior of the receptacle 36. Similar functionality is disclosed in the Related Applications and features of the receptacle 36, the cinch band 42, and the revision tulip assembly 34 may be similar to features disclosed in the Related Applications. While one size of tulip assembly 34 and receptacle 36 is shown in FIGS. 2-7, it may be appreciated that the size may be varied so as to vary the spacing between the pedicles screws 24 and the revision pedicle screw 40.

In use, the surgeon accesses the area of the segment of the spine to be fused, including exposing the construct extension protrusion 32. In this case, however, the surgeon need not expose any remaining portions of the first spinal fusion construct 20. The surgeon then implants the revision pedicle screw 40 using traditional techniques and/or techniques disclosed in the Related Applications. The surgeon then selects an appropriate size of revision tulip assembly 34 to achieve a desired spacing for the new fusion, and, if necessary for the chosen embodiment, a slotted ball 38. The surgeon then places the slotted ball 38 over the construct extension protrusion 32 and locates the revision tulip assembly 34 in the surgical site so that the receptacle 36 receives the slotted ball 38 and construct extension protrusion 32 therein. At this point, there may still be a significant range of motion between the construct extension protrusion 32 and the revision tulip assembly, including poly-axial rotation and flexion and telescopic motion of the construct extension protrusion 32 within the receptacle 36. Then, the surgeon positions the pedicle and the revision tulip assembly 34 appropriately, and creates a press or interference fit between the revision tulip assembly 34 and the construct extension protrusion 32 by sliding the cinch band over the receptacle 36, and creates a press or interference fit between the revision tulip assembly 34 and the revision pedicle screw 40, such as using methods disclosed in the Related Applications. Alternately, the order of establishing press or interference fits may be reversed. In any event, when the full press or interference fits are established, the spinal segment is stabilized.

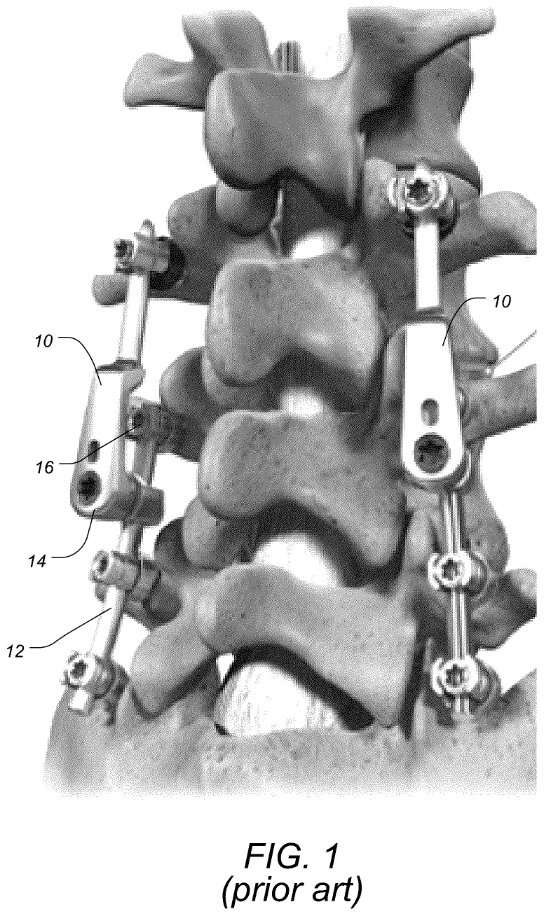

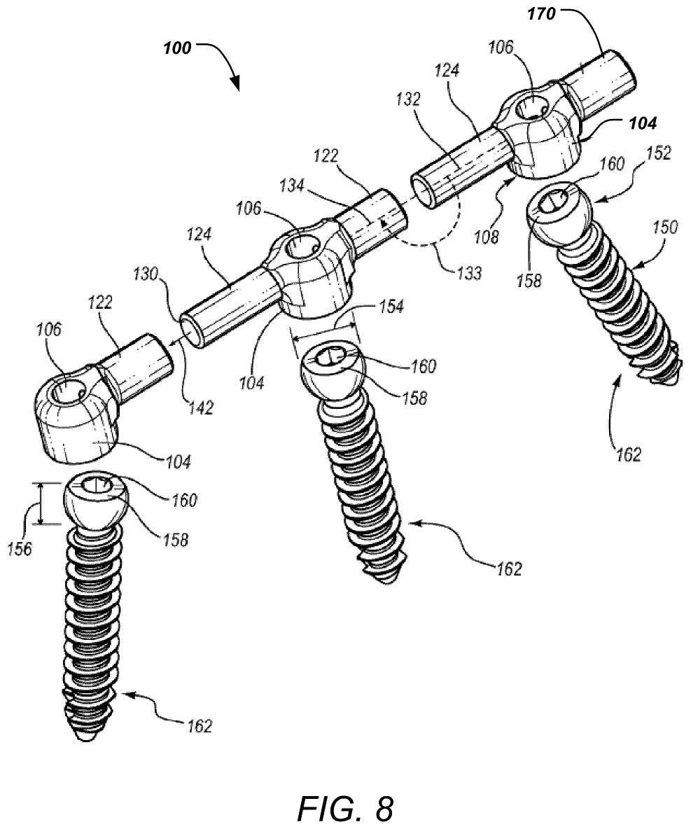

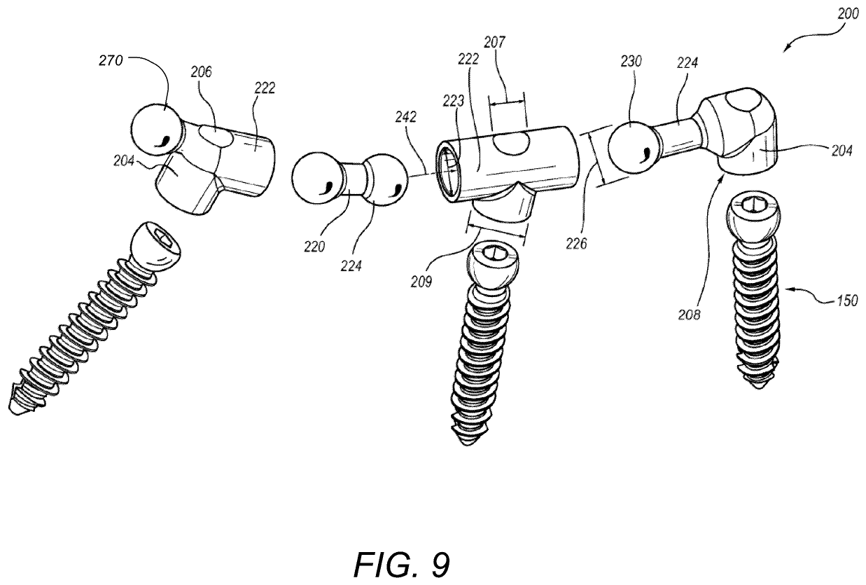

While FIGS. 2-7 show one style of the first spinal fusion construct 20, it should be appreciated that the construct extension protrusion may be added to essentially any spinal fusion construct, including those of the Related Applications, to permit revision procedures in accordance with the disclosure herein. For example, FIG. 8 shows an embodiment of a surgical screw system 100 similar to that shown in FIGS. 1-3 of U.S. Pat. No. 9,232,965 that has been modified from that system in that it has a construct extension protrusion 170 added to one end as will be described in more detail below. It should be understood that any of the embodiments disclosed in any of the Related Applications could be similarly modified, as will be illustrated and discussed in more detail with respect to FIG. 9, so the discussion with respect to FIGS. 8 and 9 is intended to be illustrative only.

In the screw system 100 of FIG. 8, various coupling assemblies are illustrated as connecting three surgical screws 150 having a threaded portion 162 together. It will be understood that various embodiments of coupling system are capable of being used with a variety of orthopedic rod placement devices, hooks, and/or surgical screws, including, but not limited to, pedicle screws and orthopedic rods used in spinal surgery. The coupling assembly is sometimes referred to as a yolk or tulip assembly.

The coupling assembly includes at least one body 104; thus, an entire coupling assembly may include a plurality of bodies 104, such as the three bodies 104 illustrated in FIG. 8. The body 104 is illustrated as having a cylindrical shape, although other shapes such as ovoid, spherical, square, and so forth may be used. The body 104 includes an upper bore 106. The body 104 also optionally includes one or more female members 122, one or more male members 124, or a combination of female members 122 and male members 124. Thus, while FIG. 8 illustrates bodies 104 that have one or two male members 124 and/or female members 122, one having skill in the art would understand that a body 104 could have 3, 4, or more male members 124 and/or female members 122, depending on the intended purpose of the coupling assembly. The male member can be rigid, semi-rigid, or flexible, depending on the application.

Referring to FIG. 8, the embodiment of the surgical screw system 100 and the coupling assembly of FIG. 8 is shown in exploded view. The surgical screws 150 include a threaded portion 162 configured to engage the bone of a patient as part of a first orthopedic surgical procedure (e.g. a first spinal fusion encompassing one or more levels). The surgical screws 150 have a head portion 152 that is received by the coupling assembly as will be discussed in further detail below. The head portion 152 has a height 156 and an outer diameter 154. The head portion 152 also includes a driving feature 160 for use with various tools to drive the surgical screw 150 into the bone. Illustrated in FIG. 8, the driving feature 160 is an Allen-key or hex-head configuration, although other configurations are known in the art, such as hex-a-lobe. The surgical screw 150 includes a screw long axis and a screw cannula. As one having skill in the art would understand, the screw cannula 164 allows the surgical screw to be maneuvered over and to receive a Kirschner wire (typically referred to as a K-wire; not illustrated), a type of guide wire used to aid in the precise placement of the surgical screw in the bone.

The male member 124 includes a long axis 132 that aligns, to a greater or lesser degree with a first axis 134 of the body 104. By align, it is meant that the long axis 132 and the first axis 134 will be approximately coincident when the male member 124 is inserted via a force 142 into the female member 122 of the body 104. As will be discussed in further detail below, it is often not practical and, sometimes, even possible, to have the long axis 132 and the first axis 134 be coincident due to the tolerances of the manufacturing and surgical placement process, simple variances in the anatomies between patients and other factors. Thus, there often exists a first angle 133 between the intersection of the long axis 132 and the first axis 134.

The body 104 optionally includes a cannula having a cannula inner diameter (or height, if the cannula is not cylindrical). The cannula is connected between the female member 122 and the upper bore 106 and provides a route for any fluids, such as blood, entrapped in the female member 122 to escape when the male member 124 is inserted into the female member 122.

The female member 122 has a female inner diameter (or height, if the female member 122 is not cylindrical) and the male member 124 has an outer diameter (or height, if the male member 124 is not cylindrical). The female member 122 is configured to receive and to provide an interference fit or press fit to the male member 124. The term interference fit shall be interpreted broadly as including the joining of any two mating parts such that one or the other (or both) parts slightly deviate in size from their nominal dimension, thereby deforming each part slightly, each being compressed, the interface between two parts creating a union of extremely high friction. The word interference refers to the fact that one part slightly interferes with the space that the other is occupying in its nominal dimension. Optionally, the interference fit can be configured to require at least 800 pounds of force to remove the male member 124 from the female member 122.

The female inner diameter 123 can be constant, or it can be sloped or have steps through the inner length of the female member 122 up until a shoulder that prevents the male member 124 from advancing any further into the female member 122. Thus, the female inner diameter may be larger than an interference fit diameter near a mouth of the female member 122, thereby allowing the male member 124 to rotate, pivot, enter, and exit freely or semi-freely within the female member 122. As the male member 124 is pressed further into the female member 122, the female inner diameter optionally necks down, becoming smaller or narrower, either smoothly or in steps, until an interference fit is achieved. Thus, it will be understood that the female member 122 optionally provides, in part, the ability to adjust the distance between bodies 104.

The shoulder optionally narrows or necks down from the female inner diameter to the cannula inner diameter, or it can optionally neck down in steps. The shoulder optionally includes a lip or ridge that prevents a male face 130 of the male member 124 from advancing beyond the shoulder.

The body or bodies 104, as noted, include an upper bore 106 having a bore inner diameter (or width, if the upper bore 106 is not cylindrical). As noted, the upper bore 106 is fluidly connected to the cannula 114. The upper bore 106 is also fluidly connected to a receptacle 108 as disclosed in more detail in the prior patent. The bore inner diameter is less than the receptacle 108 inner diameter (or width, if the receptacle 108 is not cylindrical). Optionally, the bore inner diameter 107 is sufficiently wide to accommodate a K-wire or other guiding mechanism threaded through the screw cannula 164 as discussed above.

The receptacle 108, in addition to the receptacle inner diameter, has a height and a shoulder, or screw head contact surface. As with the female member 122, the receptacle 108 is configured to receive a head portion 152 of the surgical screw 150 and to provide an interference or press fit between the receptacle 108 and the head portion 152. The receptacle inner diameter can be constant, or it can be sloped or have steps though the inner height of the receptacle 108 up until the screw head contact surface or shoulder that prevents the head portion 152 from advancing any further into the receptacle 108. Thus, the receptacle inner diameter may be larger than an interference-fit diameter near a mouth of the receptacle 108, thereby allowing the head portion 152 to rotate, enter, and exit freely or semi-freely within the receptacle 108. As the head portion 152 is pressed further into the receptacle 108, the receptacle inner diameter optionally necks down, becoming smaller or narrower, either smoothly or in steps, until an interference fit is achieved.

The body 104 and, hence, the receptacle 108 includes a second axis. The second axis may align, to a greater or lesser degree with the screw long axis 166 of the surgical screw 150, as is the case for the middle body 104 in FIG. 8. By align, it is meant that the screw long axis and the second axis will be approximately coincident when the head portion 152 of the surgical screw 150 is inserted into the receptacle 108 of the body 104. As will be discussed in further detail below, it is often not practical and, sometimes, even possible, to have the screw long axis and the second axis be coincident due to the tolerances of the manufacturing and surgical placement process, simple variances in the anatomies between patients and other factors. In other circumstances, the screw long axis intersects the second axis of the body at a second angle, as illustrated in the leftmost body 104 of FIG. 8.

The embodiment of FIG. 8 differs from the embodiment of FIGS. 1-3 of U.S. Pat. No. 9,232,965 in that it includes the construct extension protrusion 170 extending from the last or rightmost body 104 shown in FIG. 8. The construct extension protrusion 170 is approximately cylindrical, and it may have any desired diameter, though the diameter of the construct extension protrusion 170 will generally be consistent along the length of the construct extension protrusion 170 in this type of example. The construct extension protrusion 170 of this example is adapted to receive a slotted ball 38 as discussed above. While not shown in FIG. 8, an additional construct extension protrusion 170 could similarly be provided to the leftmost body 104 of FIG. 8, allowing for a later revision procedure at that end of the surgical screw system 100 as well.

FIG. 9 shows an embodiment of a surgical screw system 100 similar to that shown in FIGS. 4-6 of U.S. Pat. No. 9,232,965 that has been modified from that system in that it has a construct extension protrusion 270 added to one end as will be described in more detail below. It should be understood that any of the embodiments disclosed in any of the Related Applications could be similarly modified, so the discussion with respect to FIG. 9 (as was with FIG. 8) is intended to be illustrative only, and one of ordinary skill in the art will readily appreciate from the discussion of these two Figures how embodiments of systems from any of the related patents and applications incorporated herein (U.S. Ser. No. 11/952,709, U.S. Pat. No. 9,232,965, U.S. Ser. No. 13/455,854, or U.S. Ser. No. 14/060,757) could be modified to incorporate one or more construct extension protrusions 170 or construct extension protrusion 270 as discussed herein.

Accordingly, FIG. 9 illustrates another embodiment of a surgical screw system 200 that includes a plurality of another embodiment of coupling assemblies illustrated as connecting three surgical screws 150 having a threaded portion together. The surgical screws 150 illustrated were previously described above, thus reference should be made to that description.

The coupling assembly includes at least one body 204; thus, an entire coupling assembly may include a plurality of bodies 204, such as the three bodies 204 illustrated in FIG. 9. The body 204 is illustrated as having a cylindrical shape, although other shapes such as ovoid, spherical, square, and so forth fall within the scope of the disclosure. The body 204 includes an upper bore 206. The body 204 also optionally includes one or more female members 222, one or more male members 224, a combination of female members 222 and male members 224, and, optionally, a coupling rod 220 that includes two or more male members 224 or female members 222. (Illustrated is coupling rod 220 with male members 224, but this is just one embodiment of the coupling rod, as will be understood.) Thus, while FIG. 9 illustrates bodies 204 that have one or two male members 224 and/or female members 222, and one coupling rod 220, one having skill in the art would understand that a body 204 could have 3, 4, or more male members 224 and/or female members 222 and/or coupling rods 220, depending on the intended purpose of the coupling assembly.

As with the embodiment of FIG. 8, the male member 224 and the coupling rod 220 include a long axis that aligns, to a greater or lesser degree with a first axis of the body. By align, it is meant that the long axis and the first axis will be approximately coincident when the male member 224 (whether included with a body 204 or a coupling rod 220) is inserted via a force 242 into the female member 222 of the body 204. As will be discussed in further detail below, it is often not practical and, sometime, even possible, to have the long axis and the first axis be coincident due to the tolerances of the manufacturing and surgical placement process, simple variances in the anatomies between patients and other factors. Thus, there often exists a first angle 233 between the intersection of the long axis and the first axis. This embodiment optionally includes a male member 224 that has male face 230 that is a sphere in this instance, although other shapes and configurations, including ovoid, square, and the like, fall within the scope of the disclosure. An advantage of the male face 230 of spherical shape is that provides for a greater range of motion and possible angles 233 than may otherwise be accommodated, thus providing greater case of use and accommodation for a wider variety of factors that a surgeon might encounter while connecting the coupling assembly to a surgical screw or screws.

The body 204 optionally includes a cannula having a cannula inner diameter (or height, if the cannula is not cylindrical). The cannula is connected between the female member 22 and the upper bore 206 and provides a route for any fluids, such as blood, entrapped in the female member 222 to escape when the male member 224 is inserted into the female member 222.

The female member 222 has a female inner diameter (or height, if the female member 222 is not cylindrical) 223 and the male member 224 includes the male face 230 that has an outer diameter (or height, if the male member 224 is not cylindrical) 226. The female member 222 is configured to receive and to provide an interference fit or press fit to the male face 230 of the male member 224.

The female inner diameter 223 can be constant, or it can be sloped or have steps though the inner length of the female member 222 up until a shoulder that prevents the male face 230 of the male member 224 from advancing any further into the female member 222. Thus, the female inner diameter 223 may be larger than an interference fit diameter near a mouth of the female member 222, thereby allowing the male face 230 of the male member 224 to rotate, enter, and exit freely or semi-freely within the female member 222. As the male member 224 is pressed further into the female member 222, the female inner diameter 223 optionally necks down, becoming smaller or narrower, either smoothly or in steps, until an interference fit is achieved. Thus, it will be understood that the female member 222 optionally provides, in part, the ability to adjust the distance between bodies 204.

The shoulder optionally narrows or necks down from the female inner diameter 223 to the cannula inner diameter, or it can optionally neck down in steps. The shoulder optionally includes a lip or ridge that prevents the male face 230 of the male member 224 from advancing beyond the shoulder.

The body or bodies 204, as noted, include an upper bore 206 having a bore inner diameter (or width, if the upper bore 206 is not cylindrical) 207. As noted, the upper bore 206 is fluidly connected to the cannula 214. The upper bore 206 is also fluidly connected to a receptacle 208 that receives the screw head. The bore inner diameter 207 is less than the receptacle inner diameter (or width, if the receptacle is not cylindrical). Optionally, the bore inner diameter 207 is sufficiently wide to accommodate a K-wire or other guiding mechanism threaded through the screw cannula 164 as discussed above.

The receptacle 208, in addition to the receptacle inner diameter, has a height and a shoulder, or screw head contact surface. As with the female member 222, the receptacle 208 is configured to receive a head portion 152 of the surgical screw 150 and to provide an interference or press fit between the receptacle 208 and the head portion 152. The receptacle inner diameter can be constant, or it can be sloped or have steps though the inner height of the receptacle 208 up until the screw head contact surface or shoulder that prevents the head portion 152 from advancing any further into the receptacle 208. Thus, the receptacle inner diameter may be larger than an interference-fit diameter near a mouth of the receptacle 208, thereby allowing the head portion 152 to rotate, pivot, enter, and exit freely or semi-freely within the receptacle 208. As the head portion 252 is pressed further into the receptacle 208, the receptacle inner diameter optionally necks down, becoming smaller or narrower, either smoothly or in steps, until an interference fit is achieved.

The body 204 and, hence, the receptacle 208 includes a second axis. The second axis may align, to a greater or lesser degree with the screw long axis 166 of the surgical screw 150, as is the case for the middle body 204 in FIG. 9. By align, it is meant that the screw long axis and the second axis will be approximately coincident when the head portion 152 of the surgical screw 150 is inserted into the receptacle 208 of the body 204. It is often not practical and, sometime, even possible, to have the screw long axis and the second axis be coincident due to the tolerances of the manufacturing and surgical placement process, simple variances in the anatomies between patients and other factors. In other circumstances, the screw long axis intersects the second axis of the body at a second angle, as illustrated in the left most body 204 of FIG. 9, for which the prior art could not easily accommodate or easily allow.

The embodiment of FIG. 9 differs from the embodiment of FIGS. 4-6 of U.S. Pat. No. 9,232,965 in that it includes the construct extension protrusion 270 extending from the last or leftmost body 204 shown in FIG. 9. The construct extension protrusion 270 includes an approximately spherical portion, and it may have any desired diameter. The construct extension protrusion 270 of this example is not used with a slotted ball 38, as the spherical portion is already present. While not shown in FIG. 9, an additional construct extension protrusion 270 could similarly be provided to the rightmost body 204 of FIG. 9, allowing for a later revision procedure at that end of the surgical screw system 200 as well.

The construct extension protrusion 170 of FIG. 8 and the construct extension protrusion 270 of FIG. 9 illustrate that such construct extension protrusions 170, 270 may be unitarily formed with their respective bodies 104, 204. Accordingly, it may be said that construct extension protrusions may be unitarily formed with tulip assemblies such as any tulip assembly or coupling assembly disclosed in the related patents and applications that have been incorporated herein by reference.

Embodiments of the present invention provide a simple-to-use and low-profile construct extender for revision surgery. It attaches to the existing hardware at the existing rod end.

The required surgical exposure is reduced because access can be contained within the to-be-fused segment(s).

The novel extender provides a stronger, more reliable connection.

The unique method of locking provides a lower bulk implant.

A spherical ball can connect the new hardware to the existing hardware. This connection can be poly-axial and can be telescopic. Locking the new hardware can make rigid the mate to the existing hardware.

The connection between new and old does not have to employ a spherical ball. An interference fit about the old rod can be accomplished with other geometries and with fewer components. The locking ring and spherical ball could be eliminated and the new hardware could simply be pressed onto the end of the existing rod to create an interference fit mate between the old and the new. However, the spherical ball and the locking ring simplify the sizing of the new construct.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

1. A revision system for extending spinal fusion implants in situ, comprising:
a tulip assembly adapted to be fixedly secured to a pedicle screw as part of a second spinal fusion procedure adjacent a site of a prior spinal fusion procedure, the tulip assembly comprising a receptacle adapted to be fixedly secured to a construct extension protrusion of a spinal fusion construct from the prior spinal fusion procedure via a press or interference fit.

2. The revision system as recited in claim 1, wherein the receptacle comprises a slot that permits the receptacle to flex open or closed.

3. The revision system as recited in claim 2, wherein the tulip assembly comprises a cinch band adapted to be moved over a portion of the receptacle comprising the slot so as to force the receptacle to a closed position forming the press or interference fit over the construct extension protrusion.

4. The revision system as recited in claim 1, further comprising a slotted ball having a aperture sized to receive the construct extension protrusion therein, the slotted ball being adapted to be placed over the construct extension protrusion and to fit within the receptacle as the press or interference fit is achieved.

5. The revision system as recited in claim 1, wherein before the interference or press fit is achieved, the construct extension protrusion may be received within the receptacle while a range of motion is maintained between the construct extension protrusion and the tulip assembly selected from the group consisting of poly-axial rotation and flexion and telescopic motion of the construct extension protrusion within the receptacle.

6. A revision system for extending spinal fusion implants in situ, comprising:
a tulip assembly adapted to be fixedly secured to a pedicle screw as part of a first spinal fusion procedure; and
a construct extension protrusion extending from the tulip assembly in a direction roughly parallel to a next level of a spine adjacent to a level of the spine to be fused as part of the first spinal fusion procedure.

7. The revision system as recited in claim 6, wherein the construct extension protrusion is unitarily formed with the tulip assembly.

8. The revision system as recited in claim 6, further comprising an additional tulip assembly adapted to be fixedly secured to an additional pedicle screw as part of a second spinal fusion procedure, the additional tulip assembly comprising a receptacle adapted to be fixedly secured to the construct extension protrusion via a press or interference fit.

9. The revision system as recited in claim 8, wherein the receptacle comprises a slot that permits the receptacle to flex open or closed.

10. The revision system as recited in claim 9, wherein the additional tulip assembly comprises a cinch band adapted to be moved over a portion of the receptacle comprising the slot so as to force the receptacle to a closed position forming the press or interference fit over the construct extension protrusion.

11. The revision system as recited in claim 8, further comprising a slotted ball adapted to be placed over the construct extension protrusion and to fit within the receptacle before or as the press or interference fit is achieved.

12. The revision system as recited in claim 8, wherein before the interference or press fit is achieved, the construct extension protrusion may be received within the receptacle while a range of motion is maintained between the construct extension protrusion and the additional tulip assembly selected from the group consisting of:
poly-axial rotation and flexion; and
telescopic motion of the construct extension protrusion within the receptacle.

13. A method for performing spinal fusion to reduce surgical exposure required for any necessary future revision surgery, the method comprising:
assembling in situ a spinal fusion construct adapted to fuse one or more levels of the spine, wherein, upon assembly, a terminal end of the construct comprises a construct extension protrusion extending from the construct in a direction roughly parallel to a next level of a spine adjacent to a level of the spine fused as part of a spinal fusion procedure in which the spinal fusion construct is used.

14. A method as recited in claim 13, further comprising performing spinal fusion while reducing surgical exposure at a site of a previous spinal fusion construct, the method comprising:
exposing a terminal end of the previous spinal fusion construct, the previous spinal fusion construct comprising a construct extension protrusion extending from the construct in a direction roughly parallel to a next level of a spine adjacent to a level of the spine fused as part of a prior spinal fusion procedure;
securing a pedicle screw to a next pedicle of the spine; and
securing an extension construct between the pedicle screw and the construct extension protrusion, wherein the extension construct is secured to the construct extension protrusion via a press fit or interference fit.
